(19) United States
(12) Patent Application Publication
(10) Pub. No.: US 2019/0259488 A1
(43) Pub. Date: Aug. 22, 2019

(54) PHYTOCHEMICAL INTAKE TRACKING SYSTEM AND METHOD OF USE

(71) Applicant: Elleni Zemenu, Arlington, TX (US)

(72) Inventor: Elleni Zemenu, Arlington, TX (US)

(21) Appl. No.: 16/283,687

(22) Filed: Feb. 22, 2019

Related U.S. Application Data
(60) Provisional application No. 62/633,893, filed on Feb. 22, 2018.

(51) Int. Cl. G16H 20/60; G09B 19/00

(52) U.S. Cl. CPC G16H 20/60; G09B 19/0092

(57) ABSTRACT

A phytochemical tracking system includes a server to receive and process information, the server having a database; a computing device to communicate with the server; and a phytochemical tracking platform to be accessed via the computing device, the phytochemical tracking platform having a food intake portal to receive consumption data from a user, the consumption data relating to food eaten by the user; and a tracking portal to compare the consumption data to a phytochemical data within the database, the tracking portal to provide the user with a visual representation of one or more phytochemical levels as determined via the comparison of consumption data to the phytochemical data. Further, the system includes the providing of information related to the phytochemical effect on health conditions.

### BACKGROUND

### 1. Field of the Invention

The present invention relates generally to health monitoring systems, and more specifically, to a food intake monitoring system for providing a user with information regarding their intake of phytochemicals from plant based foods.

### 2. Description of Related Art

Food intake monitoring systems are well known in the art and are effective means to provide data on which to base diet altering decisions. For example, FIG. 1 depicts a flowchart 101 of a conventional food intake monitoring system. Conventionally, users track and record food intake, thereby providing a means to determine information such as calorie intake, fat content, carbohydrates, etc., as shown with boxes 103, 105. The user can further track their health and exercise to make informed decisions regarding their diet, as shown with boxes 107, 109.

One of the problems commonly associated with method 101 is lacking information. For example, it is becoming increasingly known that phytochemicals, as are obtained via eating plant matter, can play a valuable role in persons health. Conventional methods lack appropriate means to track the intake of phytochemicals and thereby provide data on which a user can alter their diet more effectively.

It should be understood that phytochemicals are chemical compounds produce by plants, such as vegetables, fruits, grains, herbs, legumes, seeds, spices, and nuts. These chemicals can vary the color, taste, and smell of food, but also can provide many benefits, including exertion of anti-oxidant, anti-bacterial, antimicrobial, anti-adhesion, antiproliferative, anti-fungal, anti-inflammatory, anti-estrogenic properties and etc. Therefore, it is beneficial to monitor intake of such phytochemicals for improved health.

Accordingly, although great strides have been made in the area of health monitoring systems, many shortcomings remain.

### DESCRIPTION OF THE DRAWINGS

The novel features believed characteristic of the embodiments of the present application are set forth in the appended claims. However, the embodiments themselves, as well as a preferred mode of use, and further objectives and advantages thereof, will best be understood by reference to the following detailed description when read in conjunction with the accompanying drawings, wherein:

FIG. 1 is a flowchart of a common method of monitoring food intake;

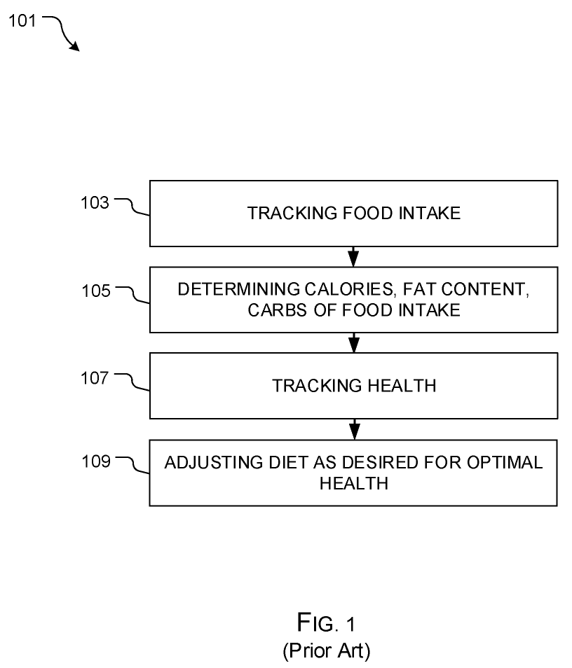

FIG. 2 is a simplified schematic of a phytochemical tracking system in accordance with a preferred embodiment of the present application; and

FIG. 3 is a flowchart of the method of use of the system of FIG. 2.

While the system and method of use of the present application is susceptible to various modifications and alternative forms, specific embodiments thereof have been shown by way of example in the drawings and are herein described in detail. It should be understood, however, that the description herein of specific embodiments is not intended to limit the invention to the particular embodiment disclosed, but on the contrary, the intention is to cover all modifications, equivalents, and alternatives falling within the spirit and scope of the present application as defined by the appended claims.

### DETAILED DESCRIPTION OF THE PREFERRED EMBODIMENT

Illustrative embodiments of the system and method of use of the present application are provided below. It will of course be appreciated that in the development of any actual embodiment, numerous implementation-specific decisions will be made to achieve the developer's specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

The system and method of use in accordance with the present application overcomes one or more of the above-discussed problems commonly associated with conventional food intake tracking systems. Specifically, the present invention provides a means to compare a person's food intake to determine their approximate phytochemical intake, and provide food recommendations thereon. In addition, the system of the present invention provides a means for a user to receive knowledge regarding the benefits of various phytochemicals. These and other unique features of the system and method of use are discussed below and illustrated in the accompanying drawings.

The system and method of use will be understood, both as to its structure and operation, from the accompanying drawings, taken in conjunction with the accompanying description. Several embodiments of the system are presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless described otherwise.

The preferred embodiment herein described is not intended to be exhaustive or to limit the invention to the precise form disclosed. It is chosen and described to explain the principles of the invention and its application and practical use to enable others skilled in the art to follow its teachings.

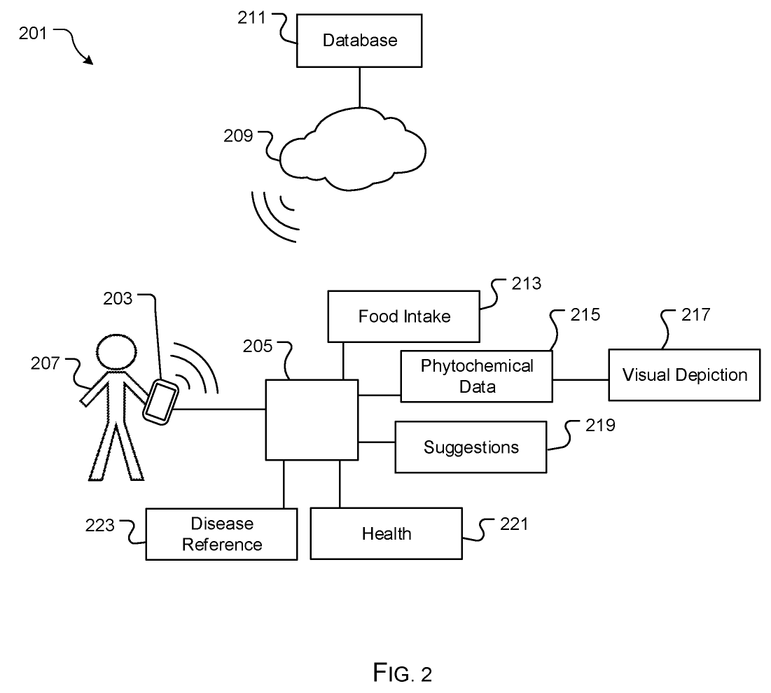

Referring now to the drawings wherein like reference characters identify corresponding or similar elements throughout the several views, FIG. 2 depicts a simplified schematic of a phytochemical tracking system 201 in accordance with a preferred embodiment of the present application. It will be appreciated that system 201 overcomes one or more of the above-listed problems commonly associated with conventional food tracking systems.

In the contemplated embodiment, system 201 includes a computing device 203 providing access to a phytochemical tracking platform 205 by a user 207, wherein computing device 203 can communicate with a server 209 to receive and transmit data with a database 211. It should be appreciated that the server, database, and computing device can be incorporated into a singular device, or alternatively, be separate devices configured to communicate.

Platform 205 includes a food intake portal 213 configured to receive food consumption data from user 207. IT should be appreciated that portal 213 can provide a list of foods, or access to a database of foods, thereby allowing for a user to select one or more foods that they have consumed during a time period. Platform 205 further includes a tracking portal 215, wherein the consumption data is compared to phytochemical data for each food, as is stored within database 211. It should be appreciated that various algorithms can be used to achieve accurate calculations of phytochemicals, based on known and future research. The phytochemicals consumed are then tracked and provided to the user via a visual depiction 217. In some embodiments, the visual depiction is merely one or more numerical values, however, it is contemplated that complex and detailed graphs, charts, and the like can be implemented for better monitoring and feedback.

In some embodiments, platform 205 further includes a suggestion portal 219, wherein a user is provided with suggested foods to eat based on their consumption data being compared to phytochemical compositions of the foods. This can be based on a particularly disease the user is concerned with, or alternatively, based on pre-defined values being deemed optimal.

Further, platform 205 can provide additional benefits through the tracking of health information 221 by user 207. For example, the user can input information such as blood pressure, glucose levels, and/or body fat percentage, wherein the continuous input by the user creates additional data for the suggestions 219 to be provided based on.

Lastly, it is contemplated that platform 205 can include a disease reference portal 223, wherein the user can access information from database 211 relating to the benefits associated with various phytochemicals as related to diseases such as osteoporosis, arteriosclerosis, type 2 diabetes, neurodegeneration, cancer, hypertension and so forth.

It should be appreciated that one of the unique features believed characteristic of the present application is the tracking of food consumption to provide information about a person's intake of various phytochemicals. It should be appreciated that this information can provide a person with improved knowledge on how to alter their diet for optimal health.

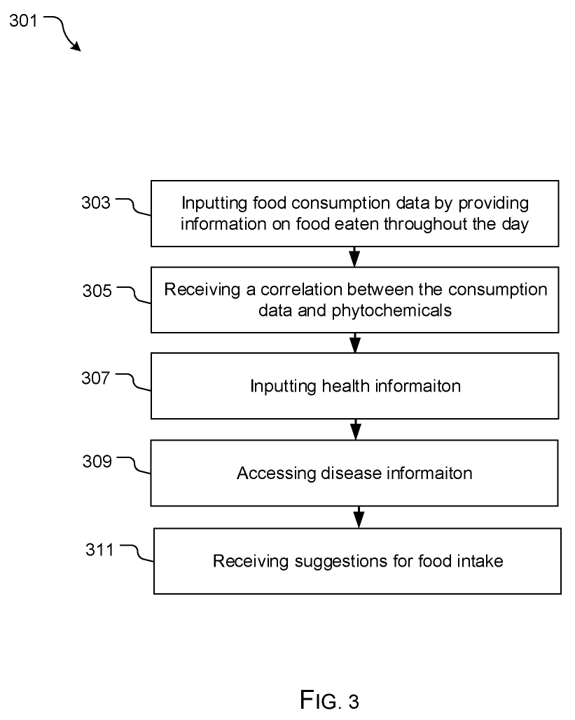

In FIG. 3, a flowchart 301 depicts a method of use of system 201. During use, the user inputs food consumption data into the platform, as shown with box 303. It should be appreciated that the platform is connected to a database of food, wherein known phytochemical compositions are provided and wherein future research can be added. A correlation between the inputted food consumption data and phytochemical data is achieved, wherein the user is provided with a visual representation of their intake, as shown with box 305. In some embodiments, the user can input health information for further tacking and monitoring, as shown with box 307. Further, as desired by the user, they can access disease information to understand better their daily, weekly, and bi-weekly needs, as shown with box 309. Based on all data received, the platform can provide the user with suggested diet changes and foods, as shown with box 311.

The particular embodiments disclosed above are illustrative only, as the embodiments may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. Although the present embodiments are shown above, they are not limited to just these embodiments, but are amenable to various changes and modifications without departing from the spirit thereof.

What is claimed is:

1. A phytochemical tracking system, comprising:
a server configured to receive and process information, the server having a database;
a computing device configured to communicate with the server; and
a phytochemical tracking platform configured to be accessed via the computing device, the phytochemical tracking platform having:
a food intake portal configured to receive consumption data from a user, the consumption data relating to food eaten by the user; and
a tracking portal configured to compare the consumption data to a plurality of phytochemical data within the database, the tracking portal configured to provide the user with a visual representation of one or more phytochemical levels as determined via the comparison of consumption data to the plurality of phytochemical data.

2. The system of claim 1, wherein the phytochemical tracking platform further comprises:
a disease reference portal configured to provide the user with access to information regarding phytochemical effects on one or more health conditions.

3. The system of claim 1, wherein the phytochemical tracking platform further comprises:
a health input portal configured to receive data from the user related to health statistics, including one or more of:
a blood pressure;
a glucose level; and
a body fat percentage level.

4. The system of claim 1, wherein the phytochemical tracking platform further comprises:
a suggestion portal configured to provide a list of food suggestions based on data received by the user.

5. A method of monitoring phytochemical intake, the method comprising:
providing access to a phytochemical tracking platform via a computing device, the computing device configured to communicate with a server to receive and transmit data;
receiving consumption data from a user related to foods eaten by the user;
correlating the consumption data to a database of a plurality of food products, the database containing phytochemical levels associated with the plurality of food products; and
providing a visual depiction of phytochemical intake associated with the user.

6. The method of claim 5, further comprising:
providing the user with disease information obtained from the database, the disease information relating to an effect of one or more phytochemicals on one or more diseases.

7. The method of claim 5, further comprising:
receiving health information from the user via the phytochemical tracking platform; and
providing one or more suggestions for food based on the health information and phytochemical data.
